(19) United States
(12) Patent Application Publication
(10) Pub. No.: US 2009/0062724 A1
(43) Pub. Date: Mar. 5, 2009

(54) SYSTEM AND APPARATUS FOR SONODYNAMIC THERAPY

(76) Inventor: Rixen Chen, Vancouver (CA)

Correspondence Address: PATTERSON & SHERIDAN, L.L.P., 3040 POST OAK BOULEVARD, SUITE 1500, HOUSTON, TX, 77056, US

(21) Appl. No.: 11/849,179

(22) Filed: Aug. 31, 2007

(51) Int. Cl. A61N 7/00

(52) U.S. Cl. 604/22

(57) ABSTRACT

The present invention relates to diffuse ultrasound along with chemical agents to treat tissue, called sonodynamic therapy (SDT), and a system for treatment using SDT that comprises a whole body ensonification apparatus and control system. The whole body ensonification may reduce the chances of missing desired tissue that may not be easily detectable or may be found throughout the body. The apparatus has a plurality of diffuse ultrasound transducers for ensonifying at least part of a chamber filled with fluid and designed to accommodate a body for treatment. The person may be treated with sono-sensitive chemical agents, which may be activated when ensonified by the apparatus.

### FIELD OF THE INVENTION

The present invention relates to diffuse ultrasound along with chemical agents to treat tissue, called sonodynamic therapy (SDT), and a system for treatment using SDT.

### BACKGROUND OF THE INVENTION

### Ultrasound

Sound waves are mechanical waves, typically generated by vibration, that propagate in a transmission medium such as air, water or human tissue. A sound wave may be categorized as follows based on its frequency:

a sound wave less than the lower limit of human hearing, typically 16 Hz, is called an infrasonic wave or infrasound;

a sound wave within the range of human hearing, typically from 16 Hz to 20 KHz, is called an acoustic wave; and

a sound wave greater than the upper limit of human hearing, typically greater than 20 KHz, is called an ultrasonic wave or ultrasound;

Ultrasonic waves can transmit higher energy than acoustic waves, and may be ideal for applications requiring the transmission of large amounts of energy, though they are often used in low energy applications as well.

In most systems, ultrasonic waves are generated by a piezoelectric material converting electrical energy to mechanical vibration and vice-versa. A common piezoelectric material is quartz crystal, but there are many other piezoelectric materials. Typically, the piezoelectric elements that generate or receive ultrasonic energy are called transducers.

In some designs, an alternating current electric signal is applied to the opposite sides of the transducer. During the positive phase of the alternating voltage, the transducer is compressed, and during the negative phase of the alternating voltage, the transducer is stretched. When alternating electric signal is applied to the transducer surfaces, the transducer makes macroscopic deformations. The frequency of the vibrations is dictated by and matches the frequency of the alternating voltage signal. This movement produces ultrasound waves with the desired frequency.

Piezoelectric materials of different thickness or compositions may have different inherent resonance frequencies. When the frequency of an alternating electric signal matches the inherent resonance frequency of a transducer, the amplitude of vibration is greatest.

While the ultrasonic wave propagates in an elastic medium, the wave's energy attenuates. There are three typical causes of attenuation:

energy losses due to expansion of the wave front,

energy losses due to scattering, and

energy losses due to absorption in the medium.

The ultrasonic wave starts with a finite amount of energy. As the ultrasonic wave spreads, that energy is distributed over a larger wave front, weakening the effect of the wave at any given point. Focusing the ultrasound, as discussed below, can have the opposite effect in the focal region.

During the course of transmission of the ultrasonic wave, if it meets the acoustic impedance and a changing interface whose dimension is equal or smaller than the wavelength, a situation different from reflection will happen. A part of the acoustic energy is dispersed to all directions (“scattering”); the remaining acoustic energy continues spreading forward.

In common tap water, bubble and impurities in the water can cause scattering and make the ultrasonic energy attenuated. Therefore, de-aerated water is used in the ultrasonic treatment in order to reduce the scattering, so that more ultrasonic energy can reach the desired focus position. Similarly, the human body is made up of mediums (skin, fat, etc . . . ) with different sound impedances, so as the sound wave travels through different tissue, some scattering will occur, which causes the wave energy to attenuate.

While the ultrasonic wave is transmitted in the tissue, its energy will also be absorbed constantly by the tissue as the wave propagates. There are at least a few principal mechanisms involved:

Viscous absorption: When the ultrasonic wave is transmitted in the tissue, vibration particles will have to overcome the viscous resistance of the particle, losing some energy in the process.

Heat conduction absorption: In the course of the transmission of sound waves, the positive and negative sound pressure in the medium will create cyclic diffusion. The temperature in positive sound pressure will rise, in negative sound pressure, the pressure will diffuse. Internal heat conduction will cause heat loss, consuming sound energy.

Molecular relaxation absorption: This is due to internal dynamics within the molecule of the medium. For instance, redistribution of the energy internal and external to the molecule, molecular structure change and chemical change, etc., can cause sound energy consumption.

The ultrasonic energy that the medium absorbs turns into heat energy, which increases the temperature. The sound intensity of the propagating wave decreases predictably as the propagation distance increases.

Current ultrasonic diagnosis imagery technology (such as b-scan diagnostic ultrasound) has been established on the foundation of the ultrasonic reflections and refractions (ultrasonic echo) from the human body. Its foundation is based on the fact that the acoustic impedance value of tissue is not uniform. Typically, when an organism suffers pathological change, its acoustic impedance value of the affected tissue changes, or the tissue itself move or changes shape, thus causing a corresponding change in the received ultrasonic echo. The ultrasonic echo thereby provides diagnostic information about pathological changes in a patient's tissue.

### Ultrasonic Treatment

Over time, a number of methods to treat tissue with ultrasound have emerged. The therapeutic potential of ultrasound was first recognized in the 1970s, when clinical physicians found that tumor cells are more sensitive to temperature than normal cells, and will die in abundance when they become hyperthermic (a rise in tissue temperature to 40-45 degrees Celsius).

It was well known at the time that when ultrasound acts on tissue, it generates heat in the focal region. The tissue absorbs ultrasonic energy and converts it to heat, and in addition heat is generated by the reflection of ultrasonic waves on different surrounding tissues back on the target tissue. It quickly became apparent to clinicians that ultrasound, with its focusing ability was an attractive tool for the targeted induction of hyperthermia in tumor cells.

The initial therapeutic applications of ultrasound to induce hyperthermia in tumor cells required long exposure to kill the cells. The early systems of treatment had a number of drawbacks. Some of these earlier systems would take an unacceptably long time to treat the target tissue, which may require the patient to be sedated or otherwise immobilized to keep the target tissue still for the long periods of time required for treatment. Some of these earlier systems would require many hours of treatment to treat a large sized tissue mass.

As the science advanced, researchers began to study the treatment of malignant tumors using high intensity ultrasound, which could generate higher temperatures. This method of ultrasound therapy is known as high intensity focused ultrasound or HIFU. HIFU allows for the thermal ablation (rise in tissue temperature to 60-85 degrees Celsius) of target tissue and has been used for the non-invasive necrosis of solid tumors. HIFU treatment is limited by the lack of specificity of thermal ablation, which indiscriminately kills both diseased and healthy cells. To avoid harming non-diseased tissue, the high intensity ultrasound must be precisely focused on the diseased target tissue.

While conducting research into the therapeutic thermal effects of ultrasound (hyperthermia and thermal ablation), researchers noticed that synergistic cytotoxic effects could be achieved through the combination of ultrasound and chemotherapy. The synergistic effects arose from the ultrasound dependent enhancement of the chemotherapy compound's cytotoxic activity. Further research led to the discovery that ultrasound could also activate non-chemotherapeutic compounds, such as hematoporphyrin.

This method of therapy based on the activation of a chemical compound by ultrasound is called sonodynamic therapy (“SDT”). The compounds that interact with ultrasound are referred to as “sonosensitizers”.

Sonodynamic Therapy

Current cancer therapy methods can diagnose and surgically remove tumor with good precision. The weakness of present cancer therapy is killing remaining metastases. It is best to kill them as early in the disease stage as possible, before they migrate and grow and damage vital tissue. The most common methods to treat cancer are chemotherapy and radiation therapy. These methods may not be suitable for many people, may not solve the problem, and may only work on some of the tumors present. Present cancer therapy may cause many harmful side effects.

Conventional scanning techniques cannot effectively detect tumors less than 5 cm. Bloodwork may not detect small cancers until much later in the disease process. The result is that doctors often can not determine if the patient needs additional treatment after surgery, can not determine if the therapy used is working, and can not determine how much treatment to give. The lack of information is most important when toxic therapies are used, because the margin for error may be low. Even if a treatment is effective, too little may not be sufficient to do the job and too much may cause harmful side effects.

Sonodynamic therapy is a method that uses ultrasound and a sono-sensitizing compound that becomes cyto-toxic when exposed to ultrasound to treat cancers. It is similar to photodynamic therapy (PDT) in its advantage of low toxicity. Unlike photodynamic therapy using light which has limited penetration, sound is able to penetrate deep into the body to reach interior cancers and tumors. Sonodynamic cancer therapy was proposed on the finding that certain sono-sensitizers are activated by acoustic cavitations caused by ultrasound and can induce substantial anti-tumor result. New generations of sono-sensitizers have been developed that are ingested and selectively absorbed by cancer cells. It is also found that a relatively low ultrasonic intensity is sufficient to activate the sono-sensitizer; it is also found that using a second frequency and superimposing it in the primary frequency may increase the treatment result. This invention is a system to deliver ultrasound to the patient to assist in the activation of sono-sensitizers, to selected parts of the body or the whole body.

Initial applications of SDT relied upon the thermal effects of ultrasound in combination with sonosensitizers that were independently cytotoxic. The sonosensitizers used in these treatments were known chemotherapeutics, such as the alkylating agents: bleomycin, adriamycin, and amphotericin. This approach to SDT has a major drawback; since both the sonosensitizer and ultrasound are independently cytotoxic, non-target tissue will likely be damaged during treatment. While the cytotoxic effect of the chemotherapeutic compound is enhanced, the whole body side effects associated with the administration of the chemotherapeutic compound remains, albeit potentially ameliorated, as does any thermal effects associated with ultrasound ensonification.

Aiming to reduce side effects and increase treatment efficiency, the modern approach of SDT has moved towards the use of the non-thermal effects of ultrasound, namely acoustic cavitation, to activate sonosensitizers that are independently non-cytotoxic. Acoustic cavitation results from alternating sound pressure, which causes the moisture contained in tissue to form tiny gas filled cavities or bubbles. Under high vibration intensity, the bubbles may explode, producing a shock wave that may cause a series of biochemical reactions and mechanical effects which in turn may activate a sonosensitizer.

In this method of treatment, both the ultrasound and sonosensitizer individually have no cytotoxic effects and will not harm healthy tissue if administered properly. In some circumstances, healthy cells flush out the sonosensitizer agent faster than cancerous cells. As such, there may a window of between 12-36 hours when it is optimal to apply ultrasound, when healthy cells have flushed out the agent, but the agent is still attached to cancerous cells. The cytotoxic effect is optimally achieved through the interaction of ultrasound and the sonosensitizer.

The mechanisms of activation of sonosensitizers that are innocuous in the absence of ultrasound are poorly understood. It has been proposed that electronic excitation of the sonosensitizer during ensonification is followed by energy transfer to oxygen, which generates highly reactive singlet molecular oxygen, which is known to have cytotoxic effects. It has also been proposed that ultrasound may interact with sonosensitizers to produce other free radicals, which may have cytotoxic properties.

Tumor-localizing porphyrin compounds were some of the first compounds used in the modern approach to SDT. Unlike the chemotherapeutic compounds, porphyrins are non-cytotoxic in the absence of ultrasound. These compounds first found application in a method of treatment that is in some ways similar to SDT; photodynamic therapy (“PDT”). PDT uses specific wavelengths of red light to activate the cytotoxic activity of photo-sensitive compounds. PDT's application to tumor treatment is restricted by the wavelength of red light, which only allows for limited tissue penetration. As a result, PDT may only treat superficial tumors that are at most 5 to 7 millimeters from the surface. Deeper tumors may be treated with interstitial irradiation; however this compromises the typically non-invasive nature of PDT.

SDT overcomes the shortcomings of PDT, because the properties of ultrasound allow it to travel easily through many centimeters of tissue, allowing for the treatment of deeper tissues. Like HIFU treatment and other treatments utilizing the thermal effects of ultrasound, focused ultrasound is used in SDT to target a specific area and protect surrounding non-diseased tissue from activated sonosensitizers. A targeted approach works well for the treatment of solid tumors, but is unsuitable for the treatment of metastases that have migrated through the body and blood born cancers such as leukemia.

PDT has been proposed for the treatment for non-solid tumors. Treatment with photo-sensitive compounds with an affinity for tumor cells allows for whole body irradiation without damage to surrounding non-diseased cells. Treatment is localized at tumor sites through the binding or internalization of photo-sensitive compounds by the tumor cells, rather then by the focusing of the light source. U.S. Pat. No. 5,484,803 (“Richter”) discloses a method of PDT, which uses photo-sensitive Benzoporphyrin derivatives, which have shown higher affinity for leukemia cells than normal non-malignant cells, in conjunction with whole body irradiation.

Sonosensitizers, like their photo-sensitive analogs, may be designed to target tumor cells. U.S. Pat. No. 6,627,664 (“Miller”) discloses the use of perylenequinone pigment derivatives conjugated with tumor binding peptides for use in localized SDT. A therapeutic amount of perylenequinone derivative is administered and allowed to distribute throughout the body. The sonosensitizers are then activated by applying ultrasound to the target area containing hyper-proliferating cells.

While cell targeted sonosensitizers have been utilized in SDT, these treatments have been limited to use in conjunction with targeted ultrasound. This targeted approach is not suitable for the treatment of diffuse cancers, such as metastases that have migrated throughout the body and cancers of the blood. For treatment of these types of cancer it would be advantageous to employ SDT that uses targeted sonosensitizers in conjunction with whole body ensonification. This type of treatment is advantageous over PDT, because it can treat deeply seated tumor cells, which would not be reached by the short wavelengths of red light delivered in PDT.

SDT involving whole body ensonification requires an apparatus that can efficiently ensonify the whole body. Traditional ultrasound apparatuses used in SDT or to induce hyperthermia or thermal ablations in diseased cells, are only capable of delivering focused ultrasound to a targeted area of the body.

In order for a whole body ensonification apparatus to work well, there are a number of considerations related to treatment that need to be addressed.

U.S. Pat. No. 7,074,427 (“Kawabata”) discloses an experimental system for measuring the effects of SDT. A test subject mouse that has had a tumor implanted in its body is immersed in a water tank filled with degassed water. The mouse is placed in a fixing device to align the implanted tumor with the focus of a focused ultrasonic transducer, which ensonifies the tumor site. Use of a focused ultrasonic transducer is undesirable for multiple reasons. Firstly, focused ultrasound limits treatment to a portion of the body where the focal point is formed. Secondly, even if multiple focused transducers were used in an attempt to ensonify the whole body, heat, cavitation, and other effects would be created at the focal points created by each transducer. Such effects are undesirable, because they can damage non-diseased tissue.

The creation of focal points can be avoided through the use of transducers that are convex in shape. Transducers of this shape produce a wide dispersion of the sound wave and greatly reduce the chance that a focal point will be created.

For the treatment to be completely effective, ultrasound must be applied to the whole body. U.S. Pat. No. 5,522,869 (“Burdette”) discloses a system for providing ultrasound thermotherapy treatment for breast cancer. A cylindrical transducer array surrounds a treatment cavity into which a diseased breast is inserted. The plurality of transducers in the array applies ultrasound to the whole breast. The apparatus disclosed in Burdette is inappropriate for use in whole body SDT, because the apparatus can only be used to treat small portions of the body. Further, the system is dependent on the thermal effects of ultrasound. This is undesirable in SDT, because there is the potential for damage to non-diseased tissue.

For the treatment to be safe and efficient the ultrasound should be applied in a controlled and reproducible manner. U.S. Pat. No. 6,450,979 (“Miwa”) discloses equipment for the lipolysis of fat from a living body by near whole body ultrasound ensonification. The equipment is composed of a bath and a single ultrasound transducer disposed on the wall of the bath. Ultrasound waves generated by the transducer travel through the water and ensonify a body positioned in the bath. Use of a single transducer is problematic for whole body ensonification, because local speckle-like irregularities occur due to the formation of standing waves. Rather than using a single transducer for full body ensonification, it would be advantageous to use a plurality of transducers that are strategically placed to fully ensonify the whole body of the patient.

There is a need to provide a HIFU system that removes or minimizes the disadvantages mentioned above.

### SUMMARY OF THE INVENTION

The invention relates to an apparatus for the whole-body ensonification of a target body for use in conjunction with a therapeutic agent for sonodynamic therapy, comprising:

a chamber for accommodating a person, the chamber at least partially filled with fluid, the fluid permitting the transmission of diffuse ultrasound; and

a first transducer and a second transducer, each producing diffuse ultrasound, the first transducer and the second transducer positioned so as to ensonify a part of the chamber containing fluid.

The invention relates to a system for the whole-body ensonification of a target body for use in conjunction with a therapeutic agent for sonodynamic therapy, comprising:

a chamber for accommodating a person, the chamber at least partially filled with fluid, the fluid permitting the transmission of diffuse ultrasound;

a first transducer and a second transducer that produce diffuse ultrasound, the first transducer and the second transducer positioned so as to ensonify a part of the chamber containing fluid;

at least one power supply, for powering the first transducer and the second transducer; and

a controller, for controlling the delivery of power from the at least one power supply to the first transducer and the second transducer.

Other features of the invention will be evident from the disclosure of several embodiments that follows.

### BRIEF DESCRIPTION OF THE DRAWINGS

Embodiments of the invention will be described by way of example and with reference to the drawings in which:

FIG. 1 is a schematic of the structure of a first embodiment according to the invention,

FIG. 2 is a schematic of the structure of a second embodiment according to the invention,

FIG. 3 is a schematic of the structure of a third embodiment according to the invention,

FIG. 4 is a schematic of the structure of a fourth embodiment according to the invention,

FIG. 5 is a detailed top view of a fifth embodiment according to the invention,

FIG. 6 is a detailed perspective view of a fifth embodiment according to the invention,

FIG. 7 is a schematic of the structure of a sixth embodiment according to the invention, and

FIG. 8 is a system schematic of an embodiment according to the invention.

### DETAILED DESCRIPTION OF THE INVENTION

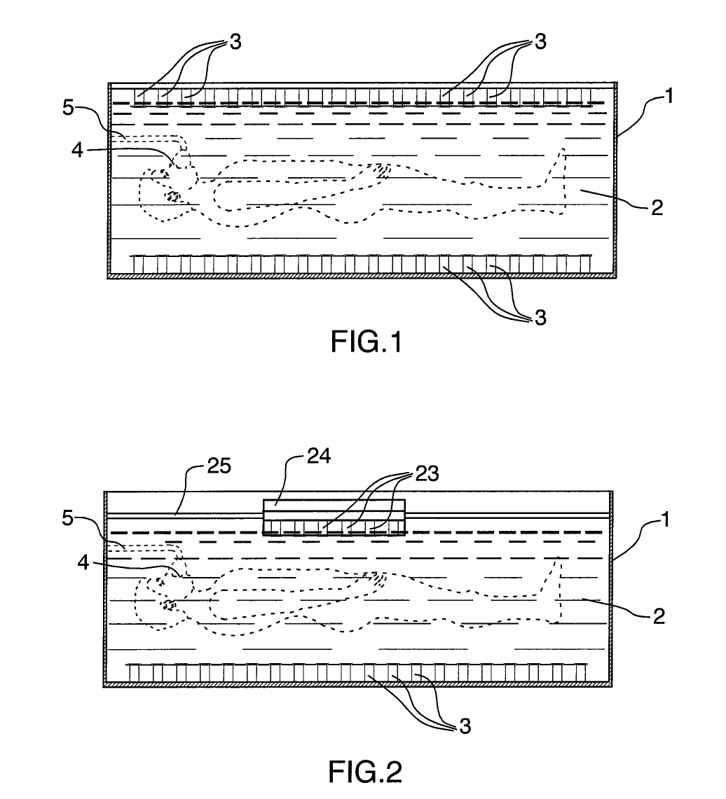

FIG. 1 is a schematic of the structure of a first embodiment according to the invention. The embodiment of the invention comprises a chamber 1 for accommodating a patient 4, the chamber containing a fluid 2 that transmits ultrasound, and a plurality of transducers 3.

The chamber 1 may be configured like a bath tub, an upright shower enclosure or any other container or enclosure capable of retaining fluid 2. The chamber may be composed of fiber glass material or any other material that is suitable to the application.

The plurality of transducers 3 may be positioned to ensonify all or part of the chamber 1. The plurality of transducers 3 are preferably in contact with the fluid 2 to allow for efficient transmission of diffuse ultrasound.

The patient 4 may be resting in the chamber partially or completely submerged in the fluid 2. A breathing apparatus 5 may be employed, but preferably the patient's face is not submerged, permitting the patient to breathe comfortably.

When the patient 4 is provided with a therapeutic agent for sonodynamic therapy, and is positioned within the chamber 1 partially or completely submerged in the fluid 2, the plurality of transducers 3 may be activated, creating a field of diffuse ultrasound throughout the chamber that activates the therapeutic agent within the patient 4.

The plurality of ultrasound transducers 3 may be strategically positioned to ensure total coverage of the patient 4 by the sound waves, both from the top and the bottom. Both the fluid level and height of the plurality of ultrasound transducers 3 may be altered, so long as the plurality of ultrasound transducers 3 are in contact with the fluid 5 when the ultrasound transducers 3 are activated.

FIG. 2 is a schematic of the structure of a second embodiment according to the invention. The embodiment of the invention comprises a chamber 1 for accommodating a patient 4, the chamber containing a fluid 2 that transmits ultrasound, and a plurality of transducers 3, some of which are moving elements 23 attached to a motional apparatus 24 running along a guide rail 25 or other movement or guidance means. The one set of moving elements 23 may be moved to provide coverage of the entire patient 4. This configuration may be used with an open top design of chamber 1, allowing easier access of the patient to the chamber. A breathing apparatus 5 may be employed, but preferably the patient's face is not submerged, permitting the patient to breathe comfortably.

The motional apparatus in any embodiment may comprise any apparatus that provides for motion in at least one direction. In some embodiments, the motional apparatus will move in two or more directions. The motional apparatus may be actuated by mechanical means, such as rails and wheels; electrical means, such as electric motors, stepper motors, or linear actuators; hydraulic or pneumatic means; by hand; or using other energetic methods to create motion. It may move along the length of the chamber, across the width of the chamber, rotate about it, and move laterally or vertically towards and away from the chamber. The movement of the motional apparatus may be to facilitate entry into or out of the chamber, to position the transducers in an optimal position to generate diffuse ultrasound that penetrates the body to a particular depth, and to permit the transducers to move so as to progressively ensonify all or a desired part of the chamber.

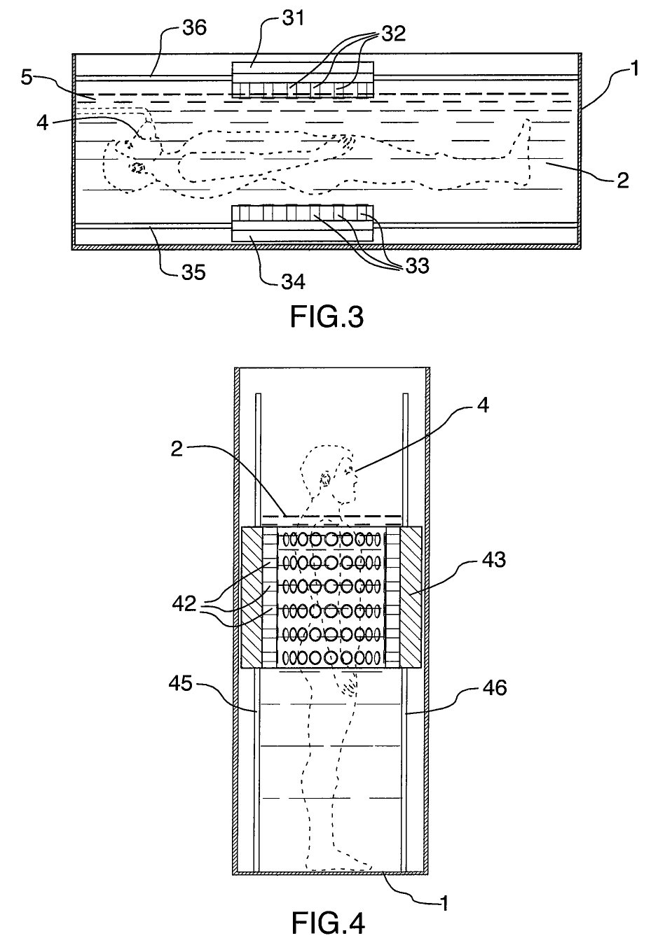

FIG. 3 is a schematic of the structure of a third embodiment according to the invention. The embodiment of the invention comprises a chamber 1 for accommodating a patient 4, the chamber containing a fluid 2 that transmits ultrasound, and a plurality of transducers, all of which are moving elements. A first set of the moving elements 32 are attached to a first motional apparatus 31 running along a guide rail 36, or the first motional apparatus may be moved or guided using other means. A second set of the moving elements 33 are attached to a second motional apparatus 34 running along a guide rail 35 or other movement or guidance means. The two sets of moving elements may provide coverage for the entire patient.

This configuration may use more than two sets of moving elements, to provide more thorough coverage of the patient 4.

A breathing apparatus 5 may be employed, but preferably the patient's face is not submerged, permitting the patient to breathe comfortably.

FIG. 4 is a schematic of the structure of a fourth embodiment according to the invention. The embodiment of the invention comprises a chamber 1 for accommodating a patient 4, the chamber containing a fluid 2 that transmits ultrasound, and a plurality of transducers, all of which are mounted on a single motional apparatus 43, which may be annular or ring shaped. The motional apparatus 43 may run along guide rails 45, 46, or may be moved or guided using other means. The motional apparatus 43 may move up and down the length of the chamber 1 while the patient stands or is supported within the fluid 2. The moving elements may provide coverage for the entire patient 4. The single motional apparatus 43 may rotate around the chamber, in an embodiment where different parts of the single motional apparatus 43 contain different types of transducers. The different types of transducers may be designed to penetrate to different depths, by varying frequency or intensity.

The patient 4 may walk into the chamber 1, which may be closed and sealed, with the patient's head exposed, or totally immersed in fluid. A breathing apparatus 5 may be employed, but preferably the patient's face is not submerged, permitting the patient to breathe comfortably. Fluid 2 is introduced into the chamber 1, and the single motional apparatus 43, which may be shaped like a circular band around the chamber, may be moved vertically up and down the enclosure 1. Motion may be provided by stepper motors, or other electromechanical means. The single motional apparatus would emit ultrasound into the fluid in the chamber 1, ensonifying the chamber 1.

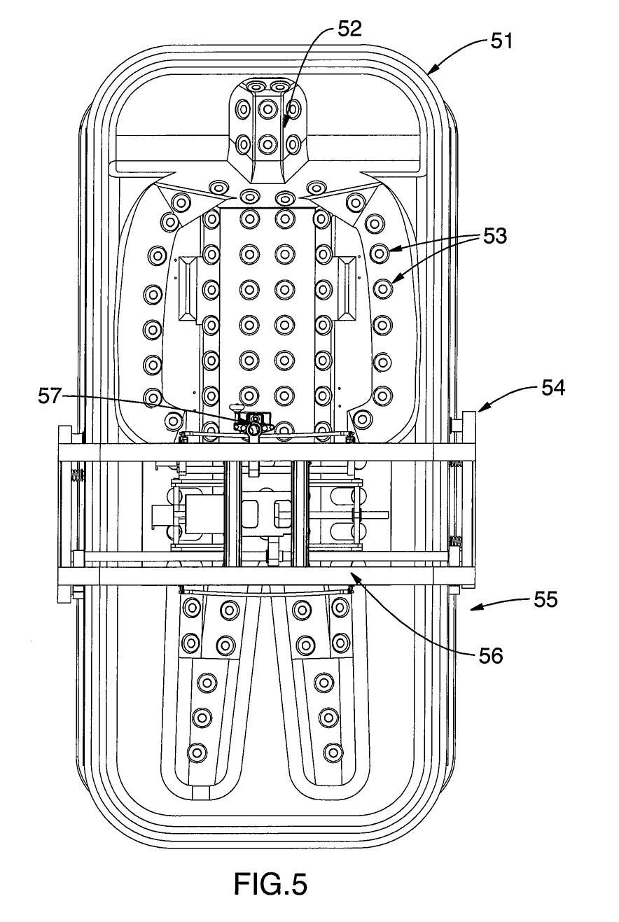

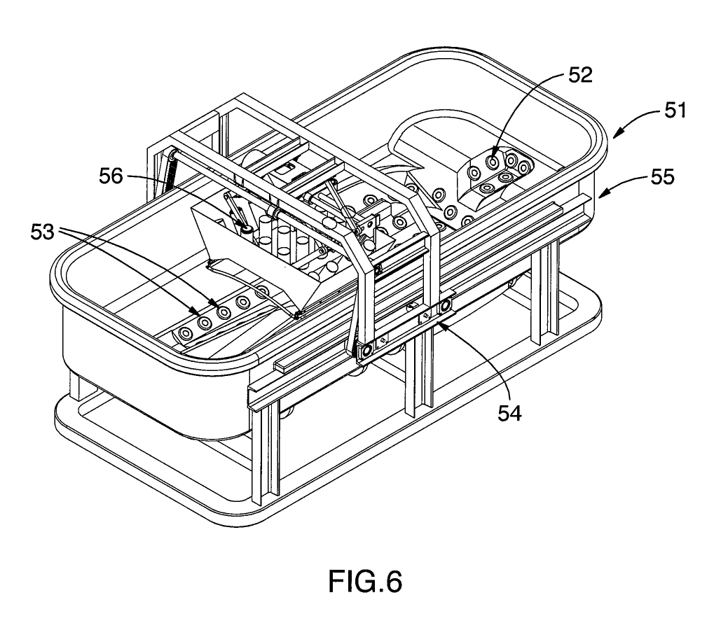

FIGS. 5 and 6 are a top view and a perspective view respectively of an embodiment of the invention, comprising a chamber 51 with an open top, having a plurality of lower elements 53 embedded in the surface of the chamber, pointed inwards. The chamber may be shaped or molded 52 to accommodate a person. A motional apparatus 54 is mounted on a set of guide rails 55, allowing movement along the length of the chamber 51. On the motional apparatus 54 are mounted at least one upper transducer element, and preferably a number of elements 56, oriented generally downwards towards the chamber 51.

A patient may lie in the chamber 51, submerged in fluid except for the face, which is exposed. The patient's head may be supported by a shaped portion of the chamber 52. The motional apparatus 54 may move lengthwise along the chamber 51 with the upper transducer elements 56 activated, in conjunction with the lower transducer elements 53, to ensonify the entire chamber or only desired portions of it.

The treatment may be carried out in sections. For example, start from the bottom (bottom one-third of the body), provide treatment, moves to the next position (say the middle of the body) and provides treatment then moves to the top (head and shoulder) and provide treatment. The procedure may be repeated.

There may be a b-scan ultrasound unit 57 which provides a practitioner ultrasound feedback during the treatment process. The b-scan ultrasound unit 57 may be mounted on the motional apparatus 54 or on the chamber, and may move independently of the motional apparatus 54. Other imaging technologies could be used instead of ultrasound.

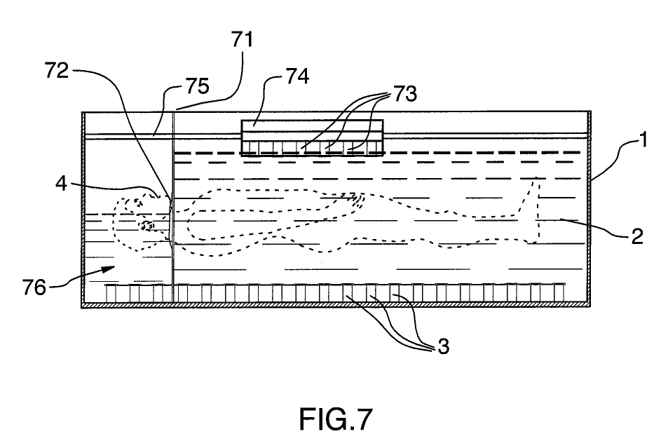

In FIG. 7, the chamber 1 is adapted to accommodate a patient 4. The chamber 1 may comprise one or more sections, each separated by a barrier or membrane 71, the membrane 71 having an opening 72 to accommodate a body part of the patient 4. In this embodiment, the patient's head is shown to be separated from the rest of the chamber 1 by the membrane 71. Fluid 2 may be introduced on one side of the membrane, into one section of the chamber 1, to immerse the patient's body, and at least partially cover a moving element 74 having at least one transducer mounted thereon 73. On the other side of the membrane 71, fluid 76 may be introduced to a different height, so as to leave the patient's face uncovered to enhance comfort and permit regular breathing.

Along the bottom of the chamber, there may be mounted a plurality of transducers 3 that produce ultrasound within all the sections of the chamber 1. Along the top, there is a motional apparatus 74, which may be adapted to run along rails 75, and upon which at least one transducer 73 may be mounted. The plurality of transducers 3 may ensonify both sections of the chamber 1, whereas at least one transducer 73 may ensonify only one section. During operation, the motional apparatus 74 passes over the patient's body, and in conjunction with the plurality of transducers 3 ensonifies the patient's body. The plurality of transducers 3 also ensonifies the patient's head.

Instead of a membrane, any barrier may be used to partially or completely separate sections of the chamber to permit differing levels of fluid in each section. In one embodiment, a ridge of flexible material forms a dam that fits snugly around the back and sides of the neck of the patient. The soft material around the neck reduces or stops the flow of fluid from one section to the other. Pumps, valves and other fluid control means may be used to control the amount and level of fluid in each section of the chamber.

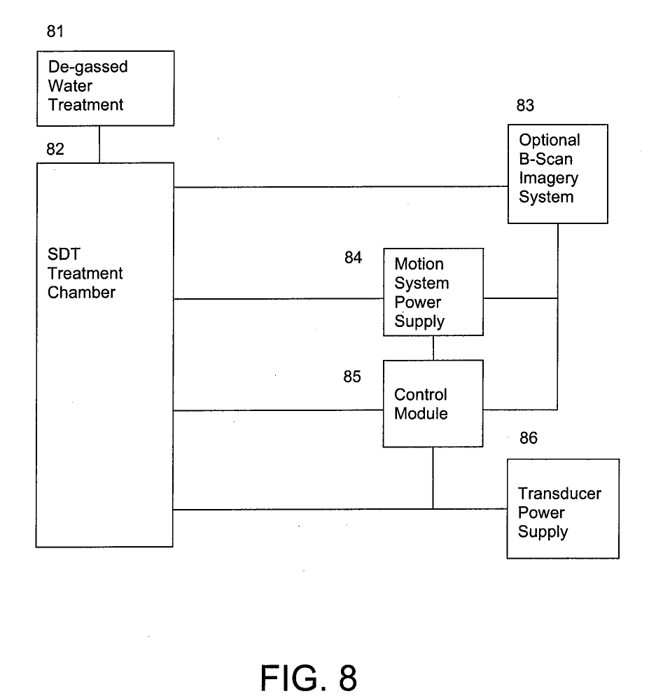

In FIG. 8, the system has an SDT treatment chamber 82 for accommodating all or part of a person, the SDT treatment chamber 82 at least partially filled with degassed fluid provided by a de-gassed water treatment apparatus 81. The SDT treatment chamber 82 has a motion system that moves at least two transducers to ensonify at least part of the SDT treatment chamber 82. The motion system may be powered by a motion system power supply 84. The transducers may be powered by at least one transducer power supply 86. A control module 85, which may be a computer, a microprocessor, an electric circuit, or any other electrical or mechanical control device, selectively actuates the power supplies 84, 86 to proceed through a pattern of treatment on all or a part of a person within the SDT treatment chamber 82. A b-scan ultrasound imagery system 83 may be used to guide treatment, confirm the presence of ultrasound within the SDT treatment chamber 82, and localize treatment on specific tissue areas. The b-scan unit 83 motion may be powered by the motion system power supply 84 and may be controlled by the control module 85. The system may employ sensors on the motional apparatus, such as an optical safety wave guide that senses distance to ensure that neither the motional apparatus nor the attached components come in contact with the patient, or scratch or hurt the patient when in operation. In one embodiment, the system uses an infrared transmitter on one side and receiver on the other side.

A chamber may be configured in any way that permits the retention of fluid and the accommodation of at least part of a person's body. The chamber may have a single opening or multiple openings, and may accommodate a chair or bench.

The target tissue within the patient may be any kind of tissue found within the body, such as cancerous cells, fat, muscle, bone, connective tissue, blood cells, specific proteins, or any other tissue that in conjunction with a sonodynamic agent has been made sensitive to diffuse ultrasound.

The system may employ mechanical safety rods or other safety systems on the bottom four sides of the motional apparatus. The safety rods detect the movement of the patient. If the patient moves beyond acceptable range, or needs to stop the treatment, the safety system is activated by the patient tripping the safety mechanism by moving or pressing against the safety bars. For example, if the treatment intensity is too strong, and the patient can not withstand the treatment, the patient will reflexively contract their muscles, pressing against the safety rods and stopping treatment immediately. The system may use non-mechanical means to detect movement, such as IR or ultrasonic sensors, instead of or in combination with mechanical means. The safety systems may be used to prevent the motional apparatus from hitting or striking the patient during use, when the apparatus is being moved in a horizontal, lateral, rotational or vertical direction.

In one embodiment, the fluid level is automatically adjusted to pre-determined depth using a water overflow valve. The water may be pumped or gravity fed into the chamber, and may be circulated to maintain a consistent temperature. The fluid temperature may adjusted according to requirement for patient comfort. Normal tap water may be used as a fluid, but de-gassed water or fluid that transmits sound well is preferable. The fluid may be degassed using any known degassing system.

The diffuse ultrasound produced may be anything within the ultrasonic range. In one embodiment, the system may be configured to produce ultrasound at frequencies greater than 20 kHz, and preferably in the range from 200 kHz to 3 MHz for applications that penetrate deep into the body. Higher frequencies, such as from 7 MHz to 15 MHz or higher can be used to treat areas closer to the skin such as muscle, fat or adipose tissue, or the skin itself. The system may be configured to produce ultrasound at more than one frequency. In the case of a system employing multiple frequencies, it is preferably that the frequencies are selected so that they minimize interference with each other, so that the waves and their phase do not create a constructive interference and increase the power of the output wave dangerously, nor create a destructive interference so that the total output wave pattern becomes too weak for the application. An example of a single frequency design uses 1 MHz; an example of a dual frequency design employing both a 660 KHz and a 1 MHz frequency.

In one embodiment, the transducers may produce a range of ultrasound that may be tuned to differing frequencies. One reason to alter the frequency of the ultrasound is to vary the depth to which the diffuse ultrasound penetrates the body. This may be useful for treating the skin or fat beneath the skin.

The system may use diffuse ultrasound transducers, which are preferably convex in shape, to produce a relatively uniform and even ultrasonic field within the fluid. The convex shape of each transducer may allow a wide field of dispersion of the sound wave, and may reduce the occurrence of focal points. Focal points create heat, which is undesirable. All that is needed is for the ultrasound to effectively activate the sono-sensitizers. Each transducer may be individually powered and controlled, and may be grouped into sections.

In an embodiment with multiple frequencies design, it is preferred to place transducers of differing frequencies adjacent to one another

In one embodiment, a number of 30 Amp, 48 VDCac power supplies are used to supply 48 VDC power to each transducer module. Each transducer module may contain its own amplifying circuit, and its own modulating circuit calibrated to the required transducer output frequency. This configuration is preferred because it may minimize EMI interference. Each transducer may be individually wired to the amplifier for individual control.

The system may be computer controlled, with options to program in the desired wave intensity, duration and continuous mode or pulse mode and pulse timing. The system may include hardware control that controls the motional apparatus, the treatment intensity and pattern, and the individual transducers' operating parameters. The system may include software systems to store and display patient information and system information.

In one embodiment, the transducers may be configured to offer continuous treatment or pulse mode treatment, using a controller.

In one embodiment, the apparatus further comprises one or more supports, handles or bars to assist an infirm or elderly patient into the treatment chamber. In another embodiment, the support is mechanized to lift or move an infirm or elderly patient into the treatment chamber.

What is claimed is:

1. An apparatus for the whole-body ensonification of a target body for use in conjunction with a therapeutic agent for sonodynamic therapy, comprising:
a. a chamber for accommodating a person, the chamber at least partially filled with fluid, the fluid permitting the transmission of diffuse ultrasound; and
b. a first transducer and a second transducer, each producing diffuse ultrasound, the first transducer and the second transducer positioned so as to ensonify one or more parts of the chamber containing fluid.

2. The apparatus of claim 1, further comprising a motional apparatus, and wherein the first transducer is located on the motional apparatus.

3. The apparatus of claim 2, wherein the chamber has an axis along its length, and the motional apparatus moves laterally along the axis.

4. The apparatus of claim 2, wherein the motional apparatus moves transversely relative to the axis.

5. The apparatus of claim 2, wherein the motional apparatus moves rotationally about the axis.

6. The apparatus of claim 1, wherein the first transducer operates at a first frequency, and the second transducer operates at a second frequency.

7. The apparatus of claim 6, wherein the first transducer is placed adjacent to the second transducer.

8. The apparatus of claim 1, wherein the first transducer and the second transducer are each shaped with a convex transducer element face to produce diffuse ultrasound.

9. The apparatus of claim 2, further comprising a safety mechanism mounted on the motional apparatus, to deactivate the first transducer and the second transducer when triggered.

10. The apparatus of claim 9, wherein the safety mechanism comprises at least one mechanical rod connected to a switch to deactivate the first transducer and the second transducer.

11. The apparatus of claim 9, wherein the safety mechanism comprises at least one sensor to deactivate the first transducer and the second transducer, the at least one sensor selected from the group consisting of: light sensors, laser sensors, infrared sensors, motion sensors, and ultrasonic sensors.

12. The apparatus of claim 1, further comprising a fluid pump, and wherein the fluid in the chamber is circulated using the fluid pump.

13. The apparatus of claim 12, wherein the amount of the fluid in the chamber is automatically adjusted by a controller to maintain a desired level of fluid.

14. The apparatus of claim 12, wherein the level of the fluid in the chamber is maintained by use of an overflow valve.

15. A system for the whole-body ensonification of a target body for use in conjunction with a therapeutic agent for sonodynamic therapy, comprising:
a. a chamber for accommodating a person, the chamber at least partially filled with fluid, the fluid permitting the transmission of diffuse ultrasound;
b. a first transducer and a second transducer that produce diffuse ultrasound, the first transducer and the second transducer positioned so as to ensonify a part of the chamber containing fluid;
c. at least one power supply, for powering the first transducer and the second transducer; and
d. a controller, for controlling the delivery of power from the at least one power supply to the first transducer and the second transducer.

16. The system of claim 15, wherein the controller independently controls the power delivered to the first transducer and the second transducer.

17. The system of claim 16, wherein the first transducer comprises a first amplifying means for amplifying the power of first transducer and a modulating means for modulating the frequency of the first transducer, and the second transducer comprises a second amplifying means for amplifying the power of second transducer and a second modulating means for modulating the frequency of the second transducer.

18. The system of claim 15, wherein the first transducer and the second transducer are operated in continuous mode.

19. The system of claim 15, wherein the first transducer and the second transducer are operated in pulse mode.

20. The system of claim 15, further comprising a motional apparatus, and wherein the first transducer is located on the motional apparatus.

21. The system of claim 20, wherein the second transducer is located on the motional apparatus.

22. The system of claim 20, wherein the chamber has an axis along its length, and the motional apparatus moves laterally along the axis.

23. The system of claim 20, wherein the motional apparatus moves transversely relative to the axis.

24. The system of claim 20, wherein the motional apparatus moves rotationally about the axis.

25. The system of claim 15, wherein the first transducer operates at a first frequency, and the second transducer operates at a second frequency.

26. The system of claim 25, wherein the first transducer is placed adjacent to the second transducer.

27. The system of claim 15, wherein the first transducer and the second transducer are shaped with a convex transducer element face to produce diffuse ultrasound.

28. The system of claim 20, further comprising a safety mechanism mounted on the motional apparatus, to deactivate the first transducer and the second transducer when triggered.

29. The system of claim 29, wherein the safety mechanism comprises at least one mechanical rod connected to a switch to deactivate the first transducer and the second transducer.

30. The system of claim 29, wherein the safety mechanism comprises at least one sensor to deactivate the first transducer and the second transducer, the at least one sensor selected from the group consisting of: light sensors, laser sensors, infrared sensors, motion sensors, and ultrasonic sensors.

31. The system of claim 15, further comprising a fluid pump, and wherein the fluid in the chamber is circulated using the fluid pump.

32. The system of claim 31, wherein the amount of the fluid in the chamber is automatically adjusted by a controller to maintain a desired level of fluid.

33. The system of claim 31, wherein the level of the fluid in the chamber is maintained by use of an overflow valve.

34. The system of claim 31, wherein the amount of the fluid in the chamber is automatically adjusted by a controller to maintain a desired level of fluid.

35. The system of claim 31, wherein the level of the fluid in the chamber is maintained by use of an overflow valve.

36. The system of claim 15, wherein each transducer is configured to vary in frequency.

37. A method for the whole-body ensonification of a target body for use in conjunction with a therapeutic agent for sonodynamic therapy using an apparatus comprising:
a. a chamber for accommodating a person, the chamber having a longitudinal axis, the chamber at least partially filled with fluid, the fluid permitting the transmission of diffuse ultrasound; and
b. a plurality of transducers that produce diffuse ultrasound, the plurality of transducers positioned so as to ensonify a part of the chamber containing fluid;
the method comprising the following steps:
i. administering a therapeutic agent for sonodynamic therapy to a patient;
ii. positioning the patient within the chamber, and submerging the patient at least partially in the fluid; and
iii. activating the plurality of transducers to produce diffuse ultrasound that penetrates the patient and triggers the therapeutic agent.

38. The method of claim 37, wherein the apparatus further comprises a motional apparatus that moves along the longitudinal axis, the motional apparatus having at least one transducer of the plurality of transducers mounted upon it, and the method comprising the following steps:
iv. moving the motional apparatus to position the at least one transducer over an untreated portion of the patient;
v. repeating steps iii. and iv. as many times as necessary to completely ensonify the desired portions of the patient.
